(19) United States
(12) Patent Application Publication
(10) Pub. No.: US 2020/0207539 A1
(43) Pub. Date: Jul. 2, 2020

(54) Containers Containing Degradable Carriers

(71) Applicant: SAINT-GOBAIN PERFORMANCE PLASTICS CORPORATION, Solon, OH (US)

(72) Inventors: Natasha Anna Boghosian, Boston, MA (US); Katie Campbell, Northborough, MA (US); Jian L. DIng, Stow, MA (US); Natalie Fekete, Marlborough, MA (US); Rachel Z. Pytel, Newton, MA (US)

(21) Appl. No.: 16/730,477

(22) Filed: Dec. 30, 2019

Related U.S. Application Data
(60) Provisional application No. 62/786,921, filed on Dec. 31, 2018.
(60) Provisional application No. 62/786,932, filed on Dec. 31, 2018.

(51) Int. Cl. B65D 85/50; C08L 27/22; C12M 1/00

(52) U.S. Cl. CPC B65D 85/50; B65D 2201/00; C12M 47/02; C08L 27/22

(57) ABSTRACT

This disclosure relates to containers and substrates comprising a fluoropolymer and having a textured inner surface and to methods for cultivating and/or isolating a biological agent using such containers and substrates. In one embodiment, the disclosure provides a container (e.g., in the form of a bag) having an outer surface and a textured inner surface, the inner surface comprising a fluoropolymer, wherein the textured inner surface comprises one or more of (a) a plurality of functional groups attached to the fluoropolymer, and (b) a pattern of spaced features, the features having an average spacing within the range of 0.1 μm to 200 μm.

### CROSS-REFERENCE TO RELATED APPLICATIONS

This application claims the benefit of priority of U.S. Provisional Patent Applications Nos. 62/786,921, filed Dec. 31, 2018; and 62/786,932, filed Dec. 31, 2018; each of which is hereby incorporated herein by reference in its entirety.

### BACKGROUND OF THE DISCLOSURE

### Field of the Disclosure

This disclosure relates generally to containers (e.g., in the form of bags) and substrates having textured surfaces. More particularly, the present disclosure relates to containers and substrates comprising a fluoropolymer and having a textured surface, and to methods for cultivating and/or isolating a biological agent using such containers and substrates.

### Technical Background

Cell culture and cell isolation are important processes in a number of applications. For example, certain cells for use in therapeutic applications (e.g., immunotherapy, regenerative medicine, etc.) are typically isolated and cultured in vitro. For example, cells such as progenitor cells and mesenchymal stem cells, and monocytes and other immune cells are present in blood in relatively low concentrations, and accordingly are typically isolated from blood and cultured in vitro. Similarly, neuronal cells, cardiomyocytes, epithelial cells, and other cells for regenerative medicine (e.g., bone repair, skin repair, pancreatic islets regeneration, etc.) can be cultured in vitro.

Fluoropolymer bags are commonly used for cell cultures. Such bags are typically inexpensive, disposable, portable and easy to use. However, fluoropolymer surfaces are typically poor adhesion substrates for anchorage-dependent cells, which require an environment comparable to their natural cell niche to survive. Moreover, differentiation of certain stem cells can be dependent on the cell's microenvironment, including the adhesion substrate.

Certain cell isolation processes also involve anchoring a desired cell to a solid support. For example, an adherent cell can selectively adhere to a substrate, e.g., an inner surface of a container such as a bag, while undesired cells remain in a suspension that can be separated from the substrate. Existing fluoropolymer bags are generally poorly suited for such purposes.

Accordingly, there remains a need for cell culture and isolation articles having surfaces to which anchorage-dependent cells can adhere.

### SUMMARY OF THE DISCLOSURE

In one aspect, the disclosure provides a container (e.g., in the form of a bag) having an outer surface and a textured inner surface, the inner surface comprising a fluoropolymer, wherein the textured inner surface comprises one or more of (a) a plurality of functional groups attached to the fluoropolymer, and (b) a pattern of spaced features, the features having an average spacing within the range of 0.1 μm to 200 μm.

In another aspect, the disclosure provides a method for cultivating a biological agent, comprising incubating the biological agent in the container as described herein.

In another aspect, the disclosure provides a method for isolating a biological agent, comprising providing a container as described herein, the container containing the biological agent and a first aqueous medium, the agent adhered to the inner surface of the container; and removing (e.g., decanting) the first aqueous medium from the container to remove an off-target agent suspended therein.

In another aspect, the disclosure provides a substrate having a textured surface, the surface comprising a fluoropolymer; wherein the textured surface comprises a pattern of spaced features, the features having an average spacing within the range of 0.1 μm to 200 μm.

In another aspect, the disclosure provides a method for cultivating a biological agent, comprising incubating the biological agent in contact with the textured surface of the substrate as described herein.

Other aspects of the disclosure will be apparent to the person of ordinary skill in the art in view of the disclosure herein.

### BRIEF DESCRIPTION OF THE DRAWINGS

FIG. 1 provides a schematic top-down (top) and cross-sectional (bottom) view of a bag according to one embodiment of the disclosure.

FIG. 2 provides a schematic top-down view of bags according to certain embodiments of the disclosure.

FIG. 3 provides schematic top views of surfaces having patterns of spaced features

### DETAILED DESCRIPTION

In certain aspects, the disclosure provides a container (e.g., in the form of a bag) having an outer surface and a textured inner surface, the inner surface comprising a fluoropolymer, wherein the textured inner surface comprises one or more of (a) a plurality of functional groups attached to the fluoropolymer, and (b) a pattern of spaced features, the features having an average spacing within the range of 0.1 μm to 200 μm.

The containers of the disclosure can be provided in a number of forms. One especially convenient form is a bag, e.g., formed from one or more sheets of fluoropolymeric material as described herein. The person of ordinary skill in the art will be familiar with bag structures such as those used in cell culture, and will be able to adapt conventional bag structures for use in bags and methods of the disclosure based on the description herein Of course, the person of ordinary skill in the art will appreciate that the containers of the disclosure can be provided in a number of other forms, e.g., flasks, tubes, dishes.

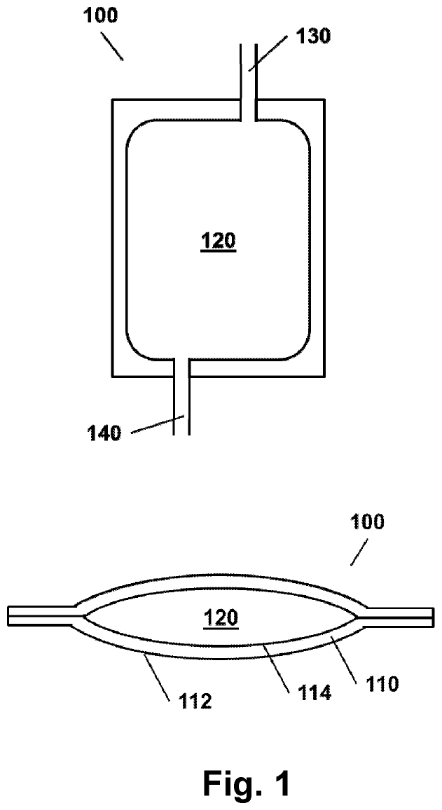

Accordingly, one aspect of the disclosure is a container (e.g., in the form of a bag) having an outer surface and a textured inner surface, the inner surface comprising a fluoropolymer. An embodiment of such a bag is shown in schematic top-down view (top) and cross-sectional view (bottom) in FIG. 1. Bag 100 of FIG. 1 includes a bag wall 110 having an outer surface 112 and an inner surface 114, and further includes ports 130 and 140, located at opposite ends of the bag for adding or removing medium (e.g., feed medium or waste medium, respectively) to or from the bag. The person of ordinary skill in the art will appreciate that the number and location of ports of the containers of the disclosure are not particularly limited, and accordingly can be positioned, for example, for convenience of use or manufacture. Bag 100 can be the product of bonding two fluoropolymer containing sheets (e.g., two sheets having a layer of fluorinated ethylene propylene sheets on an inside surface thereof) together at their edges (e.g., by laser welding, corona discharge, radiation, heat or melt lamination, etching, plasma treatment, wetting, adhesives, or combinations thereof) to form compartment 120. Ports 130 and 140 can be sealable to provide a sealed compartment 120.

The container wall can be uniform in its composition, or alternatively can include two or more distinct domains (e.g., two or more layers). For example, bonding two fluoropolymer sheets together, then coating the bonded sheets can provide an outer surface 112 differing in composition from inner surface 114. Similarly, bonding two multi-layer sheets together can provide an outer surface 112 differing in composition from inner surface 114. Multilayer sheets can be formed of both fluoropolymeric and nonfluorinated polymer materials; in such cases, a fluoropolymer layer can be provided at the inner surfaces of one or more of the multi-layer sheets. The thickness of the container, the volume of the compartment, and the shape of the container and/or compartment are not particularly limited, and can be selected for convenience of use or manufacture, and/or to suit a specific application. For example, the thickness of the container wall can be within the range of 0.0003 inches to 0.2 inches, and the volume of the compartment can be within the range of 100 mL to 100 L.

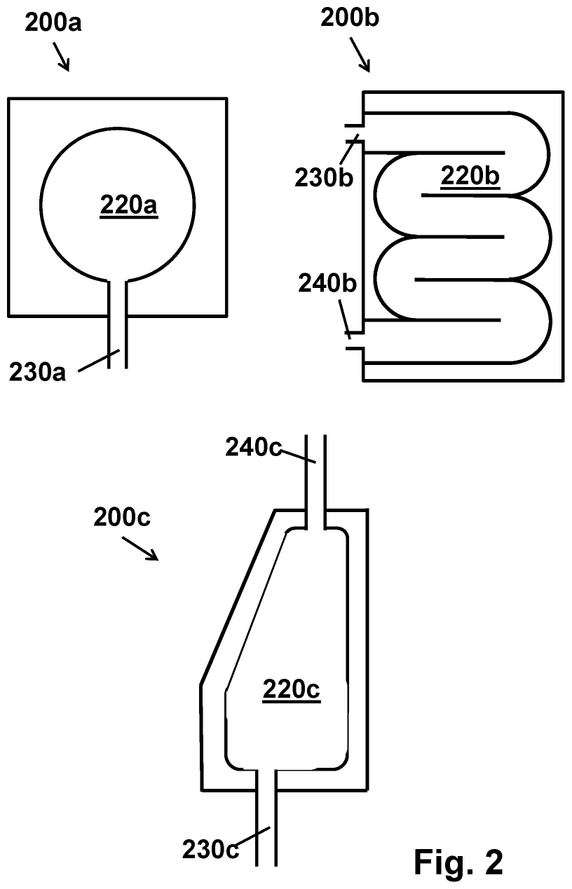

FIG. 2 shows several exemplary embodiments of configurations for culture bags suitable for use in the bags and methods of the disclosure. Bag 200a has only a single port 230a, providing access to compartment 220a. Bag 200b is in the so-called “serpentine” configuration, in which a longer path length through the system can be provided; ports 230b and 240b are connected by a serpentine path formed by serpentine-shaped compartment 220b formed by appropriate welding of the sheets forming the bag. And bag 200c has a non-rectangular shape, with a corresponding non-rectangular compartment 220c between ports 230c and 240c.

One or more of the walls of the container can be porous, and can, for example, be permeable to gases produced and consumed in a cell culture (e.g., O₂, CO₂) but impermeable to liquids (e.g., water). This can allow for passive exchange of gases across the container walls with the atmosphere to allow for respiration of cells in the container.

The containers of the disclosure are desirably formed such that there is substantially no contamination of a fluid within the container. Accordingly, it is desirable for the inner surface of the container to be formed from materials that will not leach organics into the fluid. For example, in certain embodiments as otherwise described herein, an inner surface of the container wall is formed of a polymer (e.g., a fluoropolymer such as fluorinated ethylene propylene) having a total organic carbon (TOC) in water of less than 0.1 mg/cm² (e.g., less than 0.05 mg/cm², or less than 0.05 mg/cm²). Such containers are described, e.g., in U.S. Patent Application Publications nos. 2016/0178490 and 2016/0178491, each of which is hereby incorporated herein by reference in its entirety; the person of ordinary skill in the art can, based on the description herein, adapt such containers for use in the containers and methods of the present disclosure.

As used herein, TOC is measured for a container employed in a system of the disclosure including, for example by extraction from an internal surface area of the container (with results reflected as mg/cm² are for the TOC per square centimeter of the internal area). TOC is measured according to US Pharmacopeia (USP) 643 and with equipment that utilizes a high temperature wet oxidation reaction of UV-promoted chemical oxidation (Ultra-Clean Technology Handbook; Volume 1; Ultra-Pure Water, Ohmi, Tadahiro; CRC Press, 1993, pp. 497-517). Purified water is placed in contact with the polymer for 24 hours at 70° C., for example at a ratio of 3 cm² of article surface area to 1 mL of water. The water is removed from contact with the polymer and tested in a TOC analyzer. A suitable piece of equipment is a TEKMAR DOHRMANN Model Phoenix 8000 TOC analyzer.

As noted above, in certain embodiments as otherwise described herein, the inner surface of the container further comprises a plurality of functional groups (e.g., hydrophilic functional groups) attached to the fluoropolymer. For example, the fluoropolymer of the inner surface may be functionalized with a carboxyl group, hydroxyl group, aldehyde group, carbonyl group, amine group, imine group, amide group, ester group, anhydride group, thiol group, disulfide, phenol, guanidine, thioether, indole, imidazole, or diazonium group. In certain embodiments as otherwise described herein, the functional groups include hydrophilic functional groups such as hydroxyl groups, amino groups, carbonyl groups, carboxyl groups, and phosphate groups. For example, in certain such embodiments, the functional groups include aldehyde groups. In certain embodiments as otherwise described herein, the functional groups include nitrogen-containing groups. For example, in certain such embodiments, the inner surface of the container comprises a plurality of amino functional groups.

Though the number of functional groups per unit of inner surface (i.e., functional group density) is not particularly limited, the person of ordinary skill in the art will appreciate that the functional group density can, typically, be greater than the number of biological agents (e.g., cells) that can adhere to the surface per unit of substrate surface (e.g., adhered agent density). Accordingly, in certain embodiments, the functional group density of the inner surface is greater than the adhered agent density of the inner surface (i.e., when in use). The functional group density can, for example, be selected to provide a desired water contact angle, to provide a desired surface energy for adhesion of a desired cell type to the inner surface of the container,

In certain embodiments as otherwise described herein, the functional groups at the inner surface of the container are the product of etching of the fluoropolymer. For example, in certain such embodiments, the etching comprises chemical etching, physical-mechanical etching, or plasma etching. For example, in certain embodiments, the functional groups at the inner surface are the product of chemical etching of the fluoropolymer. In certain such embodiments, the chemical etching comprises etching with sodium ammonia or sodium naphthalene. In another example, in certain embodiments, the functional groups at the inner surface are the product of physical-mechanical etching. In certain such embodiments, the physical-mechanical etching comprises sandblasting or air abrasion with silica. In another example, the functional groups at the inner surface are the product of plasma etching. In certain such embodiments, the plasma etching comprises etching with reactive plasmas such as hydrogen, oxygen, acetylene, methane, and mixtures thereof with nitrogen, argon, and helium.

In certain embodiments as otherwise described herein, the functional groups at the inner surface of the container are the product of activation of the fluoropolymer in the presence of a reactive species. For example, in certain such embodiments, the activation is plasma activation. In certain embodiments, plasma activation includes formation of reactive species on the fluoropolymer by treatment with gases such as, for example, argon, hydrogen, nitrogen, carbon dioxide, oxygen and mixtures thereof. In certain embodiments, plasma activation generates radicals and/or peroxides on a fluoropolymer. Plasma activation can, in certain embodiments, be performed at a pressure within the range of 0.1 Torr to 0.6 Torr, or within the range of 700 Torr to 760 Torr. In another example, in certain such embodiments, the activation is corona activation. In certain embodiments, corona activation includes activation of the fluoropolymer under gases such as, for example, argon, nitrogen, hydrogen, and mixtures thereof to form active sites on the fluoropolymer (e.g., susceptible to a reactive species or subsequent chemical treatment). In certain embodiments, the activation (e.g., plasma activation or corona activation) includes a reactive hydrocarbon vapor such as, for example, ketones, alcohols, p-chlorostyrene, acrylonitrile, propylene diamine, anhydrous ammonia, styrene sulfonic acid, carbon tetrachloride, tetraethylene pentamine, cyclohexyl amine, tetra isopropyl titanate, decyl amine, tetrahydrofuran, diethyl triamine, tertiary butyl amine, ethylene diamine, toluene-2,4-diisocyanate, glycidyl methacrylate, triethylene tetramine, hexane, triethyl amine, methyl alcohol, vinyl acetate, methylisopropyl amine, vinyl butyl ether, methyl methacrylate, 2-vinyl pyrrolidone, methylvinylketone, xylene, or mixtures thereof. In certain embodiments as otherwise described herein, activation (e.g., plasma activation) including a polymerizable hydrocarbon vapor selected from, for example, butylene, ethylene, glutaraldehyde, etc., provides a polymer (i.e., comprising a functional group as otherwise described herein) coated onto the fluoropolymer. The person of ordinary skill in the art will appreciate that, in certain embodiments, plasma activation including a polymerizable hydrocarbon vapor (i.e., plasma polymerization) can provide a relatively disorganized, highly cross-linked polymer coating.

In certain embodiments as otherwise described herein, the functional groups at the inner surface of the container are the product of chemically treating an activated fluoropolymer. For example, in certain such embodiments, the activated fluoropolymer is the product of plasma activation or corona activation of the fluoropolymer. In certain such embodiments, the chemical treatment is a chemical reaction such as, for example, grafting polymerization, coupling, click chemistry, condensation, or addition. In certain embodiments, the chemical treatment is grafting polymerization in solution, comprising polymerizing vinyl monomers via radical polymerization (e.g., initiated by radicals generated through plasma activation of the fluoropolymer). In certain such embodiments, the vinyl monomers are selected from, for example, acrylic acid, (meth)acrylates, (meth)alkylacrylates, styrenes, dienes, alpha-olefins, halogenated alkenes, (meth)acrylonitriles, acrylamides, N-vinyl carbazoles, N-vinyl pyrrolidones, and maleic anhydride. For example, radical polymerization of acrylic acid monomers on the fluoropolymer can, in certain embodiments, provide a dense surface of carboxyl groups. In certain embodiments, such polymerized products can be relatively organized (e.g., as compared to plasma-polymerized products).

In certain embodiments as otherwise described herein, the functional groups at the inner surface of the container are the product of coating an activated fluoropolymer. For example, in certain such embodiments, the activated fluoropolymer is the product of plasma activation or corona activation of the fluoropolymer. In certain such embodiments, the coating is wet coating, powder coating, or chemical vapor deposition. In certain embodiments, the coating is plasma-enhanced chemical vapor deposition or initiated chemical vapor deposition. In certain embodiments the coating is wet coating, for example, of one or more extracellular matrix compounds.

In certain embodiments as otherwise described herein, the textured inner surface comprises a pattern of spaced features, the features having an average spacing within the range of 0.1 μm to 200 μm. This can be in combination with the functional groups as described above, or in other embodiments, without the functional groups as described above.

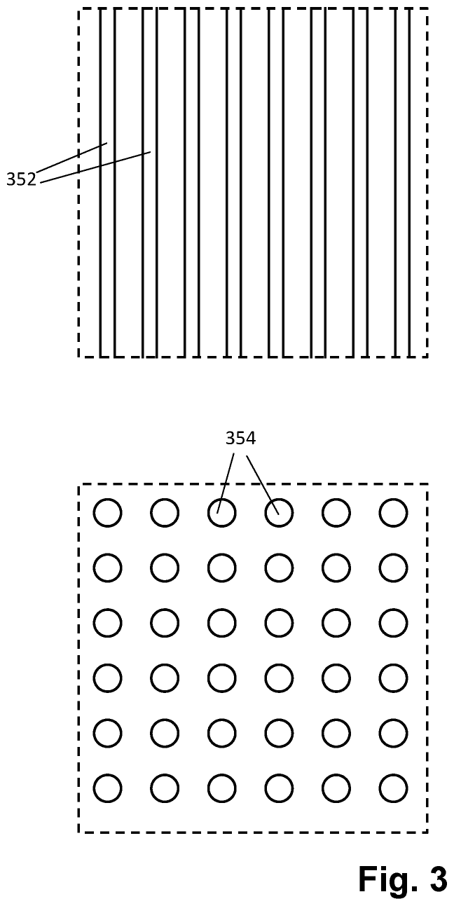

As used herein, features (i.e., of a pattern) can include any combination of raised features and depressions. The features can be elongated and have a cross-section perpendicular to the elongated axis, the cross-section having an average width and a respective average height or depth. For example, in certain embodiments as otherwise described herein, the features comprise elongated ridges or channels. The person of ordinary skill in the art will appreciate that such elongated features (e.g., ridges or channels) can have a plane of symmetry along the elongated axis. The features can also be relatively discrete (e.g., having two or more planes of symmetry). For example, in certain embodiments as otherwise described herein, the features can be pyramids (e.g., having four planes of symmetry), posts or cones (e.g., having infinite symmetrical planes), etc. FIG. 3 provides schematic top views of surfaces having patterns of spaced features; in the top example, the spaced features are ridges 352, and in the bottom example, the spaced features are humps 354.

In certain embodiments as otherwise described herein, the average slope of one or more features of the pattern (e.g., of each feature of the pattern) is less than 80° from normal (i.e., defined relative to the visible bulk surface of the substrate). For example, in certain such embodiments, the average slope of one or more features (e.g., of each feature) is less than 70°, or less than 60°, or less than 50°, or less than 40° from normal. In certain embodiments, the average slope of one or more features (e.g., of each feature) is within the range of 20° to 70°, or 20° to 60°, or 20° to 50°, or 20° to 40°, or 30° to 70°, or 40° to 70°, or 50° to 70°, or 20° to 40°, or 30° to 50°, or 40° to 60° from normal.

The person of ordinary skill in the art will appreciate that the patterned textured inner surface comprises a distribution of slopes (i.e., including the slopes of the spaced features of the pattern). The slope distribution of the patterned textured inner surfaces described herein can be non-normal, and can be unimodal or multimodal. For example, in certain embodiments as otherwise described herein, the slope distribution of the patterned textured inner surface comprises a bimodal distribution of slopes, wherein a first mode of the distribution is less than 35° from normal and a second mode of the distribution is greater than 60° from normal. In certain such embodiments, the first mode of the slope distribution is less than 30°, or less than 25°, or less than 20°, or less than 15° from normal. In certain such embodiments, the second mode of the slope distribution is greater than 65°, or greater than 70°, or greater than 75°, or greater than 80° from normal. Accordingly. in certain embodiments as otherwise described herein, at least a portion of the slope distribution (e.g., a majority of the slope distribution) is sufficiently high to provide features differentiable to a cell (e.g., an adherent cell), but sufficiently low to avoid high angles and sharp corners, which could deleteriously affect cell function.

In certain embodiments as otherwise described herein, at least 50% of the slope distribution of the patterned textured inner surface is less than 40° from normal. For example, in certain such embodiments, at least 50% of the slope distribution is less than 40° from normal. In certain such embodiments, at least 55%, or at least 60%, or at least 65% of the slope distribution is less than 40° from normal. In certain such embodiments, at least about 50% of the slope distribution is less than 35°, or less than 30°, or less than 25° from normal.

In certain embodiments, the pattern of spaced features may be, for example, the product of embossing a surface comprising a fluoropolymer, or lithographic printing onto a surface comprising a fluoropolymer. The patterning can be performed before assembling the surface into the container.

In certain embodiments as otherwise described herein, the patterned textured inner surface comprises a pattern of spaced features, the features having an average spacing within the range of 0.1 μm to 160 μm, or 0.1 μm to 140 μm, or 0.1 μm to 100 μm, or 0.1 μm to 80 μm, or 0.1 μm to 40 μm, or 1 μm to 200 μm, or 5 μm to 200 μm, or 10 μm to 200 μm, or 25 μm to 200 μm, or 50 μm to 200 μm, or 75 μm to 200 μm, or 100 μm to 200 μm, or 25 μm to 75 μm, or 50 μm to 100 μm, or 75 μm to 125 μm, or 100 μm to 150 μm, or 125 μm to 175 μm.

In certain embodiments as otherwise described herein, the pattern of spaced features comprises a first distribution of spacings. In certain such embodiments, the first distribution of spacings has a medium spacing within the range of 5 μm to 150 μm, or 5 μm to 100 μm, or 5 μm to 75 μm. In certain such embodiments, the distribution is relatively symmetric, and may assume a normal or Poisson distribution about the mean. In other embodiments, the distribution is positively or negatively skewed. In certain embodiments, the variance of the first distribution is less than 150 μm², or less than 125 μm², or less than 100 μm², or less than 75 μm², or less than 50 μm², or less than 25 μm², or less than 10 μm².

In certain embodiments as otherwise described herein, the pattern of spaced features comprises two or more distributions of spacings. For example, the features described herein can, in certain embodiments, be arranged into geometries such as circles, triangles, diamonds, etc. In such embodiments, the pattern comprises a first distribution of spacings (i.e., between the features of neighboring geometries) and a second spacing (i.e., between the features within a geometry). For example, a pattern of a feature arranged into a circle having a mean diameter of 2 μm, the circle repeated throughout the pattern with a mean center-to-center distance of 50 μm, comprises a first distribution of spacings having a first mean spacing of 50 μm and a second distribution of spacings having a second mean spacing of 2 μm. The person of ordinary skill in the art will appreciate that, in such embodiments, the average spacing of the pattern is the mean of a multimodal distribution of spacings (i.e., comprising each of the two or more distributions of spacings).

Accordingly, in certain embodiments as otherwise described herein, the pattern of spaced features comprises a first distribution of spacings having a first mean spacing within the range of 40 μm to 200 μm (e.g., within the range of 40 μm to 160 μm, or 40 μm to 120 μm) and a second distribution of spacings having a second mean spacing within the range of 0.1 μm to 40 μm (e.g., within the range of 0.5 μm to 30 μm, or 1 μm to 20 μm).

In certain embodiments as otherwise described herein, the average distance of each feature to its nearest neighboring feature is within the range of 10 μm to 100 μm. For example, in certain embodiments, the average distance of each feature to its nearest neighboring feature is within the range of 10 μm to 90 μm, or 10 μm to 80 μm, or 10 μm to 70 μm, or 10 μm to 60 μm, or 10 μm to 50 μm, or 10 μm to 40 μm, or 20 μm to 100 μm, or 30 μm to 100 μm, or 40 μm to 100 μm, or 50 μm to 100 μm, or 60 μm to 100 μm, or 20 μm to 60 μm, or 30 μm to 70 μm, or 40 μm to 80 μm, or 50 μm to 90 μm.

In certain embodiments as otherwise described herein, the pattern of spaced features is periodic. For example, in certain embodiments as otherwise described herein, the pattern comprises a repeating translation of an arrangement of features within a rectangular plane (i.e., providing a pattern of spaced features periodic along the major axes of the rectangular plane). In another example, the pattern of spaced features may be random in a first direction, but periodic in a second direction perpendicular to the first direction. As used herein, a periodic feature of a pattern along an axis comprises a distribution of spacings having a relatively low variance. For example, the spacings of a feature having a periodicity of 20 μm along a major axis can vary by at most 30% (i.e., spacings ranging from 14 μm to 26 μm), or at most 20%, or at most 10%, or at most 5%. Accordingly, in certain embodiments as otherwise described herein, the pattern of spaced features is periodic along at least one major axis of the textured inner surface. In certain such embodiments, the pattern is periodic on a scale within the range of 10 μm to 2 mm. For example, in certain embodiments as otherwise described herein, the pattern is periodic along at least one major axis of the textured inner surface, on a scale within the range of 10 μm to 1.5 mm, or 10 μm to 1 mm, or 10 μm to 750 μm, or 10 μm to 500 μm, or 10 μm to 250 μm, or 25 μm to 2 mm, or 50 μm to 2 mm, or 100 μm to 2 mm, or 250 μm to 2 mm, or 500 μm to 2 mm, or 1 mm to 2 μm, or 50 μm to 750 μm, or 50 μm to 500 μm, or 50 μm to 250 μm.

In certain embodiments as otherwise described herein, one or more features are selected from ridges and channels. In certain such embodiments, the ridges and channels have a respective average height or depth within the range of 5 nm to 1 μm. For example, in certain embodiments as otherwise described herein, one or more features are selected from ridges and channels having a respective average height or depth within the range of 25 nm to 1 μm, or 50 nm to 1 μm, or 75 nm to 1 μm, or 100 nm to 1 μm, or 150 nm to 1 μm, or 200 nm to 1 μm, or 250 nm to 1 μm, or 300 nm to 1 μm, or 350 nm to 1 μm, or 5 nm to 750 nm, or 5 nm to 500 nm, or 5 nm to 350 nm, or 5 nm to 300 nm, or 5 nm to 250 nm, or 5 nm to 200 nm, or 5 nm to 150 nm, or 5 nm to 125 nm, or 5 nm to 100 nm, or 25 nm to 750 nm, or 100 nm to 500 nm, or 150 nm to 550 nm, or 200 nm to 600 nm, or 250 nm to 650 nm, or 300 nm to 700 nm, or 350 nm to 750 nm, or 400 nm to 800 nm, or 450 nm to 850 nm, or 500 nm to 900 nm, or 550 nm to 950 nm. In certain embodiments, one or more features are selected form ridges and channels having an average width within the range of 5 nm to 500 nm. For example, in certain embodiments as otherwise described herein, one or more features are selected from ridges and channels having an average width within the range of 25 nm to 500 nm, or 50 nm to 500 nm, or 75 nm to 500 nm, or 100 nm to 500 nm, or 150 nm to 500 nm, or 200 nm to 500 nm, or 250 nm to 500 nm, or 300 nm to 500 nm, or 350 nm to 500 nm, or 5 nm to 450 nm, or 5 nm to 400 nm, or 5 nm to 350 nm, or 5 nm to 300 nm, or 5 nm to 250 nm, or 5 nm to 200 nm, or 5 nm to 150 nm, or 5 nm to 125 nm, or 5 nm to 100 nm, or 25 nm to 225 nm, or 50 nm to 250 nm, or 75 nm to 275 nm, or 100 nm to 300 nm, or 125 nm to 325 nm, or 150 nm to 350 nm, or 175 nm to 375 nm, or 200 nm to 400 nm, or 225 nm to 425 nm, or 250 nm to 450, or 275 nm to 475 nm.

In certain embodiments as otherwise described herein, one or more features are selected from features having two or more planes of symmetry such as, for example, posts or pyramids, or inverted posts or pyramids. In certain such embodiments, the features have a respective average height or depth within the range of 5 nm to 1 μm. For example, in certain embodiments as otherwise described herein, one or more features are selected from features having two or more planes of symmetry, the features having a respective average height or depth within the range of 25 nm to 1 μm, or 50 nm to 1 μm, or 75 nm to 1 μm, or 100 nm to 1 μm, or 150 nm to 1 μm, or 200 nm to 1 μm, or 250 nm to 1 μm, or 300 nm to 1 μm, or 350 nm to 1 μm, or 5 nm to 750 nm, or 5 nm to 500 nm, or 5 nm to 350 nm, or 5 nm to 300 nm, or 5 nm to 250 nm, or 5 nm to 200 nm, or 5 nm to 150 nm, or 5 nm to 125 nm, or 5 nm to 100 nm, or 25 nm to 750 nm, or 100 nm to 500 nm, or 150 nm to 550 nm, or 200 nm to 600 nm, or 250 nm to 650 nm, or 300 nm to 700 nm, or 350 nm to 750 nm, or 400 nm to 800 nm, or 450 nm to 850 nm, or 500 nm to 900 nm, or 550 nm to 950 nm. In certain embodiments, the features have an average diameter within the range of 5 nm to 500 nm. For example, in certain embodiments as otherwise described herein, one or more features are selected from features having two or more planes of symmetry, the features having an average diameter within the range of 25 nm to 500 nm, or 50 nm to 500 nm, or 75 nm to 500 nm, or 100 nm to 500 nm, or 150 nm to 500 nm, or 200 nm to 500 nm, or 250 nm to 500 nm, or 300 nm to 500 nm, or 350 nm to 500 nm, or 5 nm to 450 nm, or 5 nm to 400 nm, or 5 nm to 350 nm, or 5 nm to 300 nm, or 5 nm to 250 nm, or 5 nm to 200 nm, or 5 nm to 150 nm, or 5 nm to 125 nm, or 5 nm to 100 nm, or 25 nm to 225 nm, or 50 nm to 250 nm, or 75 nm to 275 nm, or 100 nm to 300 nm, or 125 nm to 325 nm, or 150 nm to 350 nm, or 175 nm to 375 nm, or 200 nm to 400 nm, or 225 nm to 425 nm, or 250 nm to 450, or 275 nm to 475 nm.

In certain embodiments as otherwise described herein, the pattern of spaced features includes one or more features selected from ridges or channels and one or more features selected from features having two or more planes of symmetry (e.g., posts or pyramids, or inverted posts or pyramids).

In certain embodiments as otherwise described herein, the textured inner surface has a surface roughness (i.e., regardless of whether it has a pattern of features). As used herein, surface roughness is the “vertical” variation of the height of a surface (i.e., perpendicular to the major dimensions of the surface) and the “horizontal” variation of the height of a surface (i.e., parallel to the major dimensions of the surface), excluding the respective properties of the spaced features (e.g., ridges channels, pyramids, posts, etc.) of the pattern. The person of ordinary skill in the art will appreciate that root-mean-square (RMS) roughness can describe “vertical” roughness, while “horizontal” roughness can be characterized by height-height correlation length (horizontal correlation length: ξ).

In certain embodiments as otherwise described herein, the textured inner surface has a surface roughness, the surface roughness having an RMS roughness within the range of 5 nm to 1 μm. For example, in certain such embodiments, the textured inner surface comprises a surface roughness, the surface roughness having an RMS roughness within the range of 5 nm to 900 nm, or within the range of 5 nm to 800 nm, or within the range of 5 nm to 700 nm, or within the range of 5 nm to 600 nm, or within the range of 5 nm to 500 nm, or within the range of 5 nm to 450 nm, or 5 nm to 400 nm, or 5 nm to 350 nm, or 5 nm to 300 nm, or 5 nm to 250 nm, or 5 nm to 200 nm, or 5 nm to 150 nm, or 5 nm to 100 nm, or 5 nm to 50 nm, or 5 nm to 25 nm, or 10 nm to 1 μm, or 25 nm to 1 μm, or 50 nm to 1 μm, or 100 nm to 1 μm, or 150 nm to 1 μm, or 200 nm to 1 μm, or 250 nm to 1 μm, or 300 nm to 1 μm, or 400 nm to 1 μm, or 500 nm to 1 μm, or 600 nm to 1 μm, or 700 nm to 1 μm, or 800 nm to 1 μm, or 10 nm to 750 nm, or 50 nm to 500 nm, or 100 nm to 500 nm, or 200 nm to 500 nm.

In certain embodiments as otherwise described herein, the surface roughness has a height-height correlation length within the range of 5 nm to 1 μm. For example, in certain such embodiments, the surface roughness has a height-height correlation length within the range of 5 nm to 900 nm, or 5 nm to 800 nm, or 5 nm to 700 nm, or 5 nm to 600 nm, or 5 nm to 500 nm, or within the range of 5 nm to 450 nm, or 5 nm to 400 nm, or 5 nm to 350 nm, or 5 nm to 300 nm, or 5 nm to 250 nm, or 5 nm to 200 nm, or 5 nm to 150 nm, or 5 nm to 100 nm, or 5 nm to 50 nm, or 5 nm to 25 nm, or 10 nm to 1 μm, or 25 nm to 1 μm, or 50 nm to 1 μm, or 100 nm to 1 μm, or 150 nm to 1 μm, or 200 nm to 1 μm, or 250 nm to 1 μm, or 300 nm to 1 μm, or 400 nm to 1 μm, or 500 nm to 1 μm, or 600 nm to 1 μm, or 700 nm to 1 μm, or 800 nm to 1 μm, or 10 nm to 250 nm, or 50 nm to 300 nm, or 100 nm to 350 nm, or 200 nm to 450 nm.

In certain embodiments as otherwise described herein, the textured inner surface comprises a pattern of spaced features, the features having an average spacing within the range of 0.1 μm to 200 μm, and has a surface roughness (e.g., having an RMS roughness within the range of 5 nm to 1 μm, and having a height-height correlation length within the range of 5 nm to 1 μm).

Accordingly, in various aspects and embodiments the textured inner surface comprises the pattern of spaced features and a surface roughness. Advantageously, the present inventors have determined that the combination of a pattern of spaced features (e.g., having an average spacing within the range of 0.1 μm to 200 μm) and a surface roughness (e.g., having an RMS roughness within the range of 5 nm to 1 μm, and having a height-height correlation within the range of 5 nm to 1 μm) can desirably provide a surface to which anchorage-dependent cells can adhere. The person of ordinary skill in the art will appreciate that such textured surfaces may be described in the frequency domain, using techniques such as Fourier transform, autocorrelation function or length scale fractal analysis (e.g., identifying critical features at a given spatial frequency).

Advantageously, the present inventors have determined that certain etching, activation, and treatment processes that can provide functional groups attached to the fluoropolymer can, in certain embodiments, further provide a surface having a surface roughness (e.g., within the range of 5 nm to 500 nm). In certain embodiments as otherwise described herein, the textured inner surface is the product of surface modification of the fluoropolymer. For example, in certain embodiments as otherwise described herein, the textured inner surface is the product of embossing a surface comprising a fluoropolymer, lithographic printing onto a surface comprising a fluoropolymer, or mechanically roughening a surface comprising a fluoropolymer. In another example, in certain embodiments as otherwise described herein, the textured inner surface is the product of etching (e.g., plasma etching, or physical-mechanical etching) a surface comprising a fluoropolymer.

Advantageously, the present inventors have determined that in certain embodiments, the textured inner surface of the containers described herein can be sufficiently hydrophilic to provide a compatible adhesion substrate for desired cells (e.g., anchorage-dependent cells). Accordingly, in certain embodiments, the textured inner surface has a water contact angle of less than 90°, or less than 85°, or less than 80°, or less than 75°, or less than 70°, or less than 65°, or less than 60°, or less than 55°.

A variety of fluoropolymers can be used at the inner surface of the containers as described herein. In certain embodiments as otherwise described herein, the inner surface of the container comprises a fluoropolymer selected from polytetrafluoroethylene (PTFE), perfluoroalkoxy (PFA), ethylene tetrafluoroethylene (ETFE), polyvinylidene fluoride (PVDF), polychlorotrifluoroethylene (PCTFE), ethylene chlorotrifluoroethylene (ECTFE), fluorinated ethylene propylene (FEP), ethylene fluorinated ethylene propylene (EFEP), perfluoropolyether (PFPE), modified polytetrafluoroethylene (TFM), polyvinyl fluoride (PVF), or any mixture thereof. For example, in certain embodiments as otherwise described herein, the inner surface of the container comprises fluorinated ethylene propylene. In certain embodiments as otherwise described herein, the inner surface of the container consists essentially of the fluoropolymer (e.g., fluorinated ethylene propylene) and the functional groups and aptamer sequences attached thereto. In certain embodiments as otherwise described herein, the fluoropolymer material at the inner surface of the container has a thickness of at least 0.0003 inches, at least 0.0004 inches, at least 0.0005 inches, at least 0.0006 inches, at least 0.001 inches, or at least 0.10 inches. For example, in certain such embodiments, the material comprising the inner surface of the container has a thickness within the range of 0.0003 inches to 0.2 inches, or 0.0003 inches to 0.1 inches, or 0.0005 inches to 0.08 inches, or 0.001 inches to 0.07 inches, or 0.001 inches to 0.05 inches, or 0.001 inches to 0.03 inches, or 0.001 inches to 0.018 inches, or 0.001 inches to 0.016 inches, or 0.001 inches to 0.014 inches, or 0.001 inches to 0.012 inches.

In certain embodiments as otherwise described herein, the material making up the container wall is a multilayer material, with a layer of fluoropolymer at the inner surface thereof, and a layer of another polymeric material (fluoropolymeric or otherwise) at the outer surface thereof. In certain embodiments as otherwise described herein, the material at the outer surface of the container has a thickness of at least 0.0005 inches, or at least 0.001 inches, or at least 0.005 inches, or at least 0.0075 inches, or at least 0.01 inches, or at least 0.02 inches, or at least 0.03 inches, or at least 0.04 inches, or at least 0.05 inches, or at least 0.06 inches, or at least 0.07 inches, or at least 0.08 inches, or at least 0.09 inches, or at least 0.1 inches, or at least 0.11 inches. For example, in certain such embodiments, the material at the outer surface of the container has a thickness within the range of 0.0005 inches to 0.2 inches, or 0.005 inches to 0.18 inches, or 0.01 inches to 0.16 inches, or 0.01 inches to 0.14 inches, or 0.01 inches to 0.12 inches, or 0.06 inches to 0.13 inches, or 0.09 inches to 0.126 inches.

In certain embodiments as otherwise described herein the outer surface of the container comprises a material other than a fluoropolymer. For example, in certain such embodiments, the material at the outer surface of the container comprises a thermoplastic polymer, a thermoplastic elastomer, a silicon, a rubber, or any combination thereof. Alternatively, the outer surface of the container can, in certain embodiments as otherwise described herein, comprise a fluoropolymer such as, for example, the fluoropolymer of the inner surface, or a different fluoropolymer. In certain such embodiments, the material at the inner surface and at the outer surface (i.e., the container wall) consists essentially of the fluoropolymer (e.g., fluorinated ethylene propylene), optionally with functional groups and attached at the inner surface.

In certain embodiments as otherwise described herein, the textured inner surface comprises one or more extracellular matrix (ECM) compounds attached to the fluoropolymer. For example, in certain such embodiments, the extracellular matrix compounds are selected from collagen I, poly-L-lysine, fibronectin, retronectin, hyaluronic acid, and polydopamine. In certain embodiments as otherwise described herein, one or more extracellular matrix compounds are covalently linked to a functional group of the fluoropolymer. For example, in certain such embodiments, the inner surface comprises one or more extracellular matrix proteins, attached through a peptide linkage to a carboxyl group of the fluoropolymer. In another example, the inner surface comprises one or more amine-terminated extracellular matrix proteins, attached through an imine linkage to an aldehyde group of the fluoropolymer.

In certain embodiments as otherwise described herein, the container includes a biological agent adhered to the textured inner surface. In certain embodiments as otherwise described herein, the biological agent is selected from inorganic species and organic small molecules. For example, in certain such embodiments, the biological agent is a metal ion. In other such embodiments, the biological agent is a vitamin, hormone, or peptide. In certain embodiments as otherwise described herein, the biological agent is a macromolecule such as, for example, a protein, an enzyme, or a nucleic acid (i.e., RNA or DNA). In certain embodiments as otherwise described herein, the biological agent is complex biological system such as, for example, a cell organelle (e.g., nucleus, ribosome, mitochondria, vacuole, rough endoplasmic reticulum, smooth endoplasmic reticulum, Golgi apparatus, lysosome, centrosome, vesicle, membrane) or a cell fragment. In certain embodiments as otherwise described herein, the biological agent is a bacterium or a cell group.

In certain embodiments as otherwise described herein, the biological agent is a cell. As used herein, cells adhered to a surface include cells associated with a surface sufficiently to avoid deleterious effects on cell function. The adhered cell can be relatively weakly associated with a surface (e.g., retaining a spherical shape) or strongly associated with a surface (e.g., forming a pancake-like shape on the surface). The cell can adhere directly to the fluoropolymer (e.g., comprising hydrophilic and/or nitrogen-containing functional groups) of the textured inner surface described herein (e.g., having a surface roughness within the range of 1 nm to 500 nm and/or comprising a pattern of one or more regularly spaced features, each feature having a spacing within the range of 0.1), or can interact with a protein environment (e.g., including extracellular matrix compounds) of the textured inner surface.

In certain embodiments as otherwise described herein, the cell is an anchorage-dependent cell. In certain embodiments as otherwise described herein, the cell is a blood cell or an immune cell. In certain embodiments as otherwise described herein, the cell is a stem cell, a multipotent stromal cell, a hepatocyte, a keratinocyte, an endothelial cell, an epithelial cell, or a neuron. In certain embodiments as otherwise described herein, the biological agent is a differentiated stem cell, such as, for example, a chondrocyte-like, an osteoblast-like, or an adipocyte-like differentiated stem cell. In certain embodiments as otherwise described herein, the cell is an endothelial progenitor cell, a mesenchymal stromal cell, or a loosely adherent cell such as, for example, a monocyte.

In certain embodiments as otherwise described herein, the container further includes an aqueous medium. The aqueous medium can be, for example, a cell culture medium comprising a biological agent and one or more off-target agents. As used herein, off-target agents are materials other than the biological agent including, for example, cells other than the biological agent, cell fragments, proteins, vitamins, hormones, peptides, and metal ions.

In certain embodiments as otherwise described herein, the container includes an off-target agent (e.g., a cell other than the biological agent), and no more than 20% of the off-target agent is adhered to the inner surface of the container. For example, in certain such embodiments less than 17.5%, or less than 15%, or less than 12.5%, or less than 10%, or less than 7.5%, or less than 5%, or less than 4%, or less than 3%, or less than 2%, or less than 1% of the off-target agent is adhered to the inner surface of the container.

Advantageously, the present inventors have determined that containers described herein can be suitable for cultivating biological agents that are dependent on a microenvironment comparable to a natural cell niche for survival (e.g., anchorage-dependent cells). Accordingly, another aspect of the disclosure is a method for cultivating a biological agent comprising adding the biological agent to a container as otherwise described herein. In certain such embodiments, the container contains an aqueous medium (e.g., a cell culture medium). In certain such embodiments, the biological agent is a cell and the biological agent is cultured in the container (i.e., the agent is grown, or expanded, in the aqueous medium). In certain embodiments as otherwise described herein, the cultivation provides a number of adhered cells, or a density of adhered cells, that is at least 10%, or at least 20%, or at least 30%, or at least 40%, or at least 50%, or at least 60% greater than that provided by a cultivation in an otherwise identical container having a non-modified inner surface.

The present inventors have further determined that desired cells can, in certain embodiments, selectively adhere to the textured inner surface of a container described herein. Accordingly, yet another aspect of the disclosure is a method for isolating a biological agent comprising providing a container as otherwise described herein containing a first aqueous medium and the biological agent adhered to the textured inner surface of the container. The method includes removing (e.g., decanting) the first aqueous medium to remove one or more off-target agents suspended therein.

In certain embodiments as otherwise described herein, the container contains a blood sample (e.g., a whole blood sample) or a tissue sample including the biological agent. For example, in certain such embodiments, the container contains a blood sample and the biological agent is a stem cell. In another example, in certain such embodiments, the container contains a tissue sample and the biological agent is an epithelial cell. In certain embodiments as otherwise described herein, the container further includes one or more off-target agents. For example, in certain such embodiments, the container contains a sample (e.g., a blood sample or a tissue sample) including the biological agent and one or more off-target agents (e.g., a cell other than the biological agent). In certain such embodiments, providing the container comprises adding a blood sample or a tissue sample including the biological agent to the container, and mixing the biological agent and the aqueous medium. In certain such embodiments, the biological agent and the aqueous medium are mixed for a period of time sufficient to allow the biological agent to adhere to the textured inner surface of the container. In certain embodiments as otherwise described herein, the biological agent is a cell, and the mixing is performed for a period of time and at a temperature sufficient to expand the biological agent.

Following removal of the first aqueous medium, the container as otherwise described herein can contain cells including, for example, the biological agent and optionally one or more off-target agents. Desirably, at least a portion of the cells remaining in the container can comprise the biological agent adhered to the textured inner surface of the container. For example, in certain embodiments as otherwise described herein, the biological agent is a cell, and at least 80%, or at least 82.5%, or at least 85%, or at least 87.5%, or at least 90%, or at least 92.5%, or at least 95%, or at least 97.5%, or at least 98%, or about 98.5%, or at least 99% of the cells in the container after removing the first aqueous medium are the biological agent.

Another aspect of the disclosure is a substrate having a textured surface, the surface comprising a fluoropolymer; wherein the textured surface comprises a pattern of spaced features, the features having an average spacing within the range of 0.1 μm to 200 μm. The patterned textured surface according to this aspect of the disclosure can be as described above with respect to the inner surface of the containers as described above, but need not be in a container. The substrate can include, in certain embodiments, hydrophilic functional groups as described above with respect to the inner surface of the containers. Another aspect of the disclosure is a method for cultivating a biological agent, comprising incubating the biological agent in contact with the textured surface of the substrate. Such a method can be performed as described above with respect to the containers of the disclosure.

Additional aspects of the disclosure are provided by the enumerated embodiments listed below, which can be combined in any number and in any fashion that is not technically or logically inconsistent.

Embodiment 1. A container (e.g., in the form of a bag) having an outer surface and a textured inner surface, the inner surface comprising a fluoropolymer, wherein the textured inner surface comprises one or more of (a) a plurality of functional groups attached to the fluoropolymer, and (b) a pattern of spaced features, the features having an average spacing within the range of 0.1 μm to 200 μm.
Embodiment 2. A container of embodiment 1, wherein the textured inner surface comprises the plurality of functional groups attached to the fluoropolymer.
Embodiment 3. The container of embodiment 1 or embodiment 2, wherein the functional groups include hydrophilic functional groups (e.g., hydroxyl groups, amino groups, carbonyl groups, carboxyl groups, and phosphate groups).
Embodiment 4. The container of any of embodiments 1-3, wherein the functional groups include nitrogen-containing groups (e.g., amino groups).
Embodiment 5. The container of any of embodiments 1-4, wherein the functional groups are the product of etching of the fluoropolymer.
Embodiment 6. The container of embodiment 5, wherein the chemical modification comprises chemical etching, physical-mechanical etching, or plasma etching.
Embodiment 7. The container of any of embodiments 1-4, wherein the functional groups are the product of activation of the fluoropolymer in the presence of a reactive species.
Embodiment 8. The container of embodiment 7, wherein
the activation is plasma activation or corona activation, and
the reactive species comprises one or more reactive hydrocarbon vapors.
   Embodiment 9. The container of embodiment 7, wherein
the activation is plasma activation, and
the reactive species comprises one or more polymerizable hydrocarbon vapors (e.g., selected from butylene, ethylene, and glutaraldehyde).
   Embodiment 10. The container of any of embodiments 1-4, wherein the functional groups are the product of chemically treating an activated fluoropolymer.
   Embodiment 11. The container of embodiment 10, wherein
the activated fluoropolymer is the product of plasma activation or corona activation, and
the chemical treatment is grafting polymerization, coupling, click chemistry, condensation, or addition.
   Embodiment 12. The container of any of embodiments 1-4, wherein the functional groups are the product of coating an activated fluoropolymer.
   Embodiment 13. The container of embodiment 12, wherein
the activated fluoropolymer is the product of plasma activation or corona activation, and
the coating is wet coating, powder coating, or chemical vapor deposition.
   Embodiment 14. The container of any of embodiments 1-13, wherein the textured inner surface comprises one or more extracellular matrix (ECM) compounds attached to the fluoropolymer.
   Embodiment 15. The container of embodiment 14, wherein the extracellular matrix compounds are selected from collagen I, poly-L-lysine, fibronectin, retronectin, hyaluronic acid, and polydopamine.
   Embodiment 16. The container of embodiment 14 or 15, wherein one or more extracellular matrix compounds are covalently linked to a functional group of the fluoropolymer.
   Embodiment 17. The container of any of embodiments 1-16, wherein the textured inner surface has a surface roughness, the surface roughness having an RMS roughness within the range of 5 nm to 1 μm (e.g., within the range of 5 nm to 800 nm, or within the range of 5 nm to 600 nm, or within the range of 50 nm to 500 nm).
   Embodiment 18. The container of any of embodiments 1-17, wherein the textured inner surface has a surface roughness, the surface roughness having a height-height correlation within the range of 5 nm to 1 μm (e.g., within the range of 5 nm to 800 nm, or within the range of 5 nm to 600 nm, or within the range of 50 nm to 500 nm).
   Embodiment 19. The container of embodiment 18, wherein the surface roughness is the product of one or more of etching, activation, chemical treatment, or coating of the fluoropolymer.
   Embodiment 20. The container of embodiment 19, wherein the surface roughness is the product of mechanical roughening of the fluoropolymer.
   Embodiment 21. The container of any of embodiments 1-20, wherein the textured inner surface comprises the pattern of spaced features, the features having an average spacing within the range of 0.1 μm to 200 μm.
   Embodiment 22. The container of any of embodiments 1-21, wherein the features have an average spacing within the range of 0.1 μm to 160 μm, or 0.1 μm to 120 μm, or 0.1 μm to 80 μm, or 0.1 μm to 40 μm.
   Embodiment 23. The container of any of embodiments 1-22, wherein the pattern of spaced features comprises
a first distribution of spacings having a first mean spacing within the range of 40 μm to 200 μm; and
a second distribution of spacings having a second mean spacing within the range of 0.1 μm to 40 μm.
   Embodiment 24. The container of any of embodiments 1-23, wherein the average distance of each feature to its nearest neighboring feature is within the range of 1 μm to 100 μm.
   Embodiment 25. The container of any of embodiments 1-24, wherein the pattern is periodic (e.g., on a scale within the range of 10 μm to 2 mm) along at least one major axis of the substrate.
   Embodiment 26. The container of any of embodiments 1-25, wherein one or more features are selected from ridges and channels having a respective average height or depth within the range of 5 nm to 1 μm (e.g., within the range of 50 nm to 1 μm, or within the range of 100 nm to 1 μm, or within the range of 200 nm to 1 μm) and a width within the range of 5 nm to 500 nm (e.g., within the range of 1 nm to 400 nm, or within the range of 1 nm to 300 nm).
   Embodiment 27. The container of any of embodiments 1-25, wherein one or more features are selected from features having two or more planes of symmetry, the features having a respective average height or depth within the range of 5 nm to 1 μm (e.g., within the range of 50 nm to 1 μm, or within the range of 100 nm to 1 μm, or within the range of 200 nm to 1 μm) and a width within the range of 5 nm to 500 nm (e.g., within the range of 1 nm to 400 nm, or within the range of 1 nm to 300 nm).
   Embodiment 28. The container of any of embodiments 1-27, wherein one or more features (e.g., each feature) have an average slope of less than 80° (e.g., within the range of 20° to 70°, or 20° to 50°).
   Embodiment 29. The container of any of embodiments 1-28, wherein at least 50% of a slope distribution of the textured surface is less than 40°, or less than 30°, or less than 25° from normal.
   Embodiment 30. The container of any of embodiments 1-29, wherein a slope distribution of the textured surface comprises a bimodal distribution, wherein a first mode of the slope distribution is less than 35° (e.g., less than 25°) from normal and a second mode of the slope distribution is greater than 60° (e.g., greater than 70°) from normal.
   Embodiment 31. The container of any of embodiments 1-30, wherein the pattern of spaced features is formed by embossing, etching, or lithography.
   Embodiment 32. The container of any of embodiments 1-31, wherein the textured inner surface has a water contact angle of less than 90° (e.g., less than 80°, or less than 70°, or less than 65°).
   Embodiment 33. The container of any of embodiments 1-32, wherein the fluoropolymer is polytetrafluoroethylene (PTFE), perfluoroalkoxy (PFA), ethylene tetrafluoroethylene (ETFE), polyvinylidene fluoride (PVDF), polychlorotrifluoroethylene (PCTFE), ethylene chlorotrifluoroethylene (ECTFE), fluorinated ethylene propylene (FEP), ethylene fluorinated ethylene propylene (EFEP), perfluoropolyether (PFPE), modified polytetrafluoroethylene (TFM), polyvinyl fluoride (PVF), or any mixture thereof.
   Embodiment 34. The container of any of embodiments 1-32, wherein the fluoropolymer is fluorinated ethylene propylene.
   Embodiment 35. The container of any of embodiments 1-34, comprising a biological agent adhered to the textured inner surface.
   Embodiment 36. The container of embodiment 35, wherein the biological agent is a cell (e.g., an anchorage-dependent cell).
   Embodiment 37. The container of embodiment 36, wherein the cell is a blood cell or an immune cell.
   Embodiment 38. The container of embodiment 36, wherein the cell is a stem cell, a multipotent stromal cell, a hepatocyte, a keratinocyte, an endothelial cell, an epithelial cell, a cardiomyocyte, or a neuron.
   Embodiment 39. The container of embodiment 36, wherein the cell is an endothelial progenitor cell, a mesenchymal stromal cell, or a monocyte.
   Embodiment 40. The container of any of embodiments 1-39, containing an aqueous medium.
   Embodiment 41. The container of embodiment 40, containing an off-target agent.
   Embodiment 42. The container of embodiment 41, wherein the off-target agent is a cell other than the biological agent.
   Embodiment 43. The container of embodiment 41 or 42, wherein less than 20% (e.g., less than 15%, less than 10%, less than 5%, or less than 1%) of the off-target agent is adhered to the inner surface of the container.
   Embodiment 44. A method for cultivating a biological agent, comprising incubating the biological agent in the container of any of embodiments 1-34.
   Embodiment 45. A method for isolating a biological agent, comprising
providing a container according to any of embodiments 1-34, the container containing the biological agent and a first aqueous medium, the agent adhered to the inner surface of the container; and
removing (e.g., decanting) the first aqueous medium from the container to remove an off-target agent suspended therein.
   Embodiment 46. A method of embodiment 44 or embodiment 45, wherein the biological agent is as described in any of embodiments 36-39
   Embodiment 47. A substrate having a textured surface, the surface comprising a fluoropolymer;
wherein the textured surface comprises a pattern of spaced features, the features having an average spacing within the range of 0.1 μm to 200 μm.
   Embodiment 48. The substrate of embodiment 47, wherein the features have an average spacing within the range of 0.1 μm to 160 μm, or 0.1 μm to 120 μm, or 0.1 μm to 80 μm, or 0.1 μm to 40 μm.
   Embodiment 49. The substrate of embodiment 47 or 48, wherein the pattern of spaced features comprises
a first distribution of spacings having a first mean spacing within the range of 40 μm to 200 μm; and
a second distribution of spacings having a second mean spacing within the range of 0.1 μm to 40 μm.
   Embodiment 50. The substrate of any of embodiments 47-49, wherein the average distance of each feature to its nearest neighboring feature is within the range of 1 μm to 100 μm.
   Embodiment 51. The substrate of any of embodiments 47-50, wherein the pattern is periodic (e.g., on a scale within the range of 10 μm to 2 mm) along at least one major axis of the substrate.
   Embodiment 52. The substrate of any of embodiments 47-51, wherein one or more features are selected from ridges and channels having a respective average height or depth within the range of 5 nm to 1 μm (e.g., within the range of 50 nm to 1 μm, or within the range of 100 nm to 1 μm, or within the range of 200 nm to 1 μm) and a width within the range of 5 nm to 500 nm (e.g., within the range of 1 nm to 400 nm, or within the range of 1 nm to 300 nm).
   Embodiment 53. The substrate of any of embodiments 47-51, wherein one or more features are selected from features having two or more planes of symmetry, the features having a respective average height or depth within the range of 5 nm to 1 μm (e.g., within the range of 50 nm to 1 μm, or within the range of 100 nm to 1 μm, or within the range of 200 nm to 1 μm) and a width within the range of 5 nm to 500 nm (e.g., within the range of 1 nm to 400 nm, or within the range of 1 nm to 300 nm).
   Embodiment 54. The substrate of any of embodiments 47-53, wherein one or more features (e.g., each feature) have an average slope of less than 80° (e.g., within the range of 20° to 70°, or 20° to 50°).
   Embodiment 55. The substrate of any of embodiments 47-54, wherein at least 50% of the slope distribution of the textured surface is less than 40°, or less than 30°, or less than 25° from normal.
   Embodiment 56. The substrate of any of embodiments 47-55, wherein the slope distribution of the textured surface comprises a bimodal distribution, wherein a first mode of the slope distribution is less than 35° (e.g., less than 25°) from normal and a second mode of the slope distribution is greater than 60° (e.g., greater than 70°) from normal.
   Embodiment 57. The substrate of any of embodiments 47-56, wherein the textured surface comprises a surface roughness, the surface roughness having an RMS roughness within the range of 5 nm to 1 μm (e.g., within the range of 5 nm to 800 nm, or within the range of 5 nm to 600 nm, or within the range of 50 nm to 500 nm).
   Embodiment 58. The substrate of any of embodiments 47-57, wherein the textured surface comprises a surface roughness, the surface roughness having a height-height correlation length within the range of 5 nm to 1 μm (e.g., within the range of 5 nm to 800 nm, or within the range of 5 nm to 600 nm, or within the range of 50 nm to 500 nm).
   Embodiment 59. The substrate of any of embodiments 47-58, wherein the textured surface comprises a plurality of hydrophilic functional groups attached to the fluoropolymer.
   Embodiment 60. The substrate of embodiment 59, wherein the hydrophilic functional groups are selected from hydroxyl groups, amino groups, carbonyl groups, carboxyl groups, and phosphate groups.
   Embodiment 61. The substrate of any of embodiments 47-60, wherein the textured surface has a water contact angle of less than 90° (e.g., less than 80°, or less than 70°, or less than 65°).
   Embodiment 62. The substrate of any of embodiments 47-61, wherein the textured surface comprises one or more extracellular matrix compounds attached to the fluoropolymer.
   Embodiment 63. The substrate of embodiment 62, wherein the extracellular matrix compounds are selected from collagen I, poly-L-lysine, fibronectin, retronectin, hyaluronic acid, and polydopamine.
   Embodiment 64. The substrate of any of embodiments 47-63, wherein the fluoropolymer is polytetrafluoroethylene (PTFE), perfluoroalkoxy (PFA), ethylene tetrafluoroethylene (ETFE), polyvinylidene fluoride (PVDF), polychlorotrifluoroethylene (PCTFE), ethylene chlorotrifluoroethylene (ECTFE), fluorinated ethylene propylene (FEP), ethylene fluorinated ethylene propylene (EFEP), perfluoropolyether (PFPE), modified polytetrafluoroethylene (TFM), polyvinyl fluoride (PVF), or any mixture thereof.
   Embodiment 65. The substrate of any of embodiments 47-63, wherein the fluoropolymer is fluorinated ethylene propylene.
   Embodiment 66. The substrate of any of embodiments 47-65, comprising a biological agent adhered to the textured surface.
   Embodiment 67. The substrate of embodiment 66, wherein the biological agent is a cell (e.g., an anchorage-dependent cell).
   Embodiment 68. The substrate of embodiment 67, wherein the cell is a blood cell or an immune cell.
   Embodiment 69. The substrate of embodiment 67, wherein the cell is a stem cell, a multipotent stromal cell, a hepatocyte, a keratinocyte, an endothelial cell, an epithelial cell, a cardiomyocyte, or a neuron.
   Embodiment 70. The substrate of embodiment 67, wherein the biological agent is a differentiated stem cell (e.g., chondrocyte-like, osteoblast-like, adipocyte-like).
   Embodiment 71. A method for cultivating a biological agent, comprising incubating the biological agent in contact with the textured surface of the substrate of any of embodiments 47-65.
   Embodiment 72. A method of embodiment 71, wherein the biological agent is as described in any of embodiments 67-70

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice. Thus, before the disclosed processes and devices are described, it is to be understood that the aspects described herein are not limited to specific embodiments, apparati, or configurations, and as such can, of course, vary. It is also to be understood that the terminology used herein is for the purpose of describing particular aspects only and, unless specifically defined herein, is not intended to be limiting.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All methods described herein can be performed in any suitable order of steps unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As will be understood by one of ordinary skill in the art, each embodiment disclosed herein can comprise, consist essentially of or consist of its particular stated element, step, ingredient or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, with a precision that is typical in the art.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Some embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Numerous references have been made to patents and printed publications throughout this specification. Each of the cited references and printed publications are individually incorporated herein by reference in their entirety.

Furthermore, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A container having an outer surface and a textured inner surface, the inner surface comprising a fluoropolymer, wherein the textured inner surface comprises one or more of (a) a plurality of functional groups attached to the fluoropolymer, and (b) a pattern of spaced features, the features having an average spacing within the range of 0.1 μm to 200 μm.

2. A container of claim 1, wherein the textured inner surface comprises the plurality of functional groups attached to the fluoropolymer.

3. The container of claim 1, wherein the functional groups include hydroxyl groups, amino groups, carbonyl groups, carboxyl groups, or phosphate groups.

4. The container of claim 1, wherein the functional groups include amino groups.

5. The container of claim 1, wherein the textured inner surface comprises one or more extracellular matrix (ECM) compounds attached to the fluoropolymer.

6. The container of claim 5, wherein the extracellular matrix compounds are selected from collagen I, poly-L-lysine, fibronectin, retronectin, hyaluronic acid, and polydopamine.

7. The container of claim 1, wherein the textured inner surface has a surface roughness, the surface roughness having an RMS roughness within the range of 5 nm to 1 μm.

8. The container of claim 1, wherein the textured inner surface has a surface roughness, the surface roughness having a height-height correlation within the range of 5 nm to 1 μm.

9. The container of claim 1, wherein the textured inner surface comprises the pattern of spaced features, the features having an average spacing within the range of 0.1 μm to 200 μm.

10. The container of claim 1, wherein the features have an average spacing within the range of 0.1 μm to 160 μm.

11. The container of claim 1, wherein the pattern of spaced features comprises
a first distribution of spacings having a first mean spacing within the range of 40 μm to 200 μm; and
a second distribution of spacings having a second mean spacing within the range of 0.1 μm to 40 μm.

12. The container of claim 1, wherein the average distance of each feature to its nearest neighboring feature is within the range of 1 μm to 100 μm.

13. The container of claim 1, wherein the pattern is periodic on a scale within the range of 10 μm to 2 mm along at least one major axis of the substrate.

14. The container of claim 1, wherein one or more features are selected from ridges and channels having a respective average height or depth within the range of 5 nm to 1 μm and a width within the range of 5 nm to 500 nm.

15. The container of claim 1, wherein one or more features are selected from features having two or more planes of symmetry, the features having a respective average height or depth within the range of 5 nm to 1 μm and a width within the range of 5 nm to 500 nm

16. The container of claim 1, wherein one or more features have an average slope of less than 80°.

17. The container of claim 1, wherein at least 50% of a slope distribution of the textured surface is less than 40° from normal.

18. The container of claim 1, wherein a slope distribution of the textured surface comprises a bimodal distribution, wherein a first mode of the slope distribution is less than 35° from normal and a second mode of the slope distribution is greater than 60° from normal.

19. The container of claim 1, wherein the textured inner surface has a water contact angle of less than 65°.

20. The container of claim 1, wherein the fluoropolymer is polytetrafluoroethylene (PTFE), perfluoroalkoxy (PFA), ethylene tetrafluoroethylene (ETFE), polyvinylidene fluoride (PVDF), polychlorotrifluoroethylene (PCTFE), ethylene chlorotrifluoroethylene (ECTFE), fluorinated ethylene propylene (FEP), ethylene fluorinated ethylene propylene (EFEP), perfluoropolyether (PFPE), modified polytetrafluoroethylene (TFM), polyvinyl fluoride (PVF), or any mixture thereof.

21. The container of claim 1, comprising an anchorage-dependent cell adhered to the textured inner surface.

22. The container of claim 21, wherein the cell is a blood cell or an immune cell, a stem cell, a multipotent stromal cell, a hepatocyte, a keratinocyte, an endothelial cell, an epithelial cell, a cardiomyocyte, a neuron, an endothelial progenitor cell, a mesenchymal stromal cell, or a monocyte.

23. The container of claim 1, wherein less than 20% of an off-target agent is adhered to the inner surface of the container.

24. A method for cultivating a biological agent, comprising incubating the biological agent in the container of claim 1.

25. A method for isolating a biological agent, comprising
providing a container of claim 1, the container containing the biological agent and a first aqueous medium, the agent adhered to the inner surface of the container; and
removing the first aqueous medium from the container to remove an off-target agent suspended therein.

26. A substrate having a textured surface, the surface comprising a fluoropolymer;
wherein the textured surface comprises a pattern of spaced features, the features having an average spacing within the range of 0.1 μm to 200 μm.
